(19) United States
(12) Patent Application Publication
(10) Pub. No.: US 2023/0381259 A1
(43) Pub. Date: Nov. 30, 2023

(54) DEVELOPMENT OF BIOCONVERSION PROCESS FOR FUNCTIONAL BLACK SOYBEAN POWDER BIOCONVERTED USING ENZYME GROUP DERIVED FROM BACILLUS BACTERIA AND USE THEREOF

(71) Applicant: KOOKMINBIO, CORP., Jeonju-si, Jeollabuk-do (KR)

(72) Inventor: Moon-Hee SUNG, Seoul (KR)

(73) Assignee: KOOKMINBIO, CORP., Jeonju-si, Jeollabuk-do (KR)

(21) Appl. No.: 18/027,619

(22) PCT Filed: Sep. 23, 2021
(86) PCT No.: PCT/KR2021/012930

§ 371 Date: Mar. 21, 2023

(30) Foreign Application Priority Data
Sep. 21, 2020 (KR) 10-2020-0121670
Sep. 17, 2021 (KR) 10-2021-0124802

(51) Int. Cl. A61K 36/48; A23J 1/14; A23L 33/185; A61K 35/742; A61P 39/06; C12N 1/20

(52) U.S. Cl. CPC A61K 36/48; A23J 1/148; A23L 33/185; A61K 35/742; A61P 39/06; C12N 1/205; A61K 2236/15; A61K 2236/19; C12R 2001/07

(57) ABSTRACT

The present invention relates to a method for preparing bioconversion powder having various improved physiological activity functions, by treatment with an enzyme solution derived from a Bacillus polyfermenticus strain, and provides a method for preparing bioconversion powder using a fermentation broth of a Bacillus polyfermenticus strain, by confirming that low-molecular-weight amino acids, peptides and various functional ingredients were formed by treating whole soy milk with a fermentation broth mixed with various degrading enzymes and peptide-synthesizing enzymes derived from the Bacillus polyfermenticus strain.

### TECHNICAL FIELD

The present disclosure relates to a method of preparing black soybean bioconversion powder with enhanced functional and nutritional properties, and more specifically, to a method of preparing black soybean bioconversion powder with various physiological functions enhanced by treating an enzyme solution derived from a Bacillus polyfermenticus strain.

### BACKGROUND ART

Containing proteins, fats, and various functional components that are good for the body, soybeans are ideal food which is nutritionally excellent and very important and essential in the diet. Moreover, in recent years, with newly known physiological functions such as anti-cancer properties and immune enhancement, the nutritional value of soybeans as a functional food is increasing gradually. However, although soybeans play an important role as a nutrient source of protein and fat, densified tissues make digestion and absorption very difficult. In addition, due to specific smell, poorly digestible carbohydrates, and physiological inhibitors such as trypsin inhibitors, problems concerning digestion and absorption may occur when misprocessed, leading to side effects such as diarrhea.

Soy milk, one of the major processed foods of soybeans, is a representative soybean processed product with increased soybean protein utility rate, is known as a functional nutritional drink since it is rich in soybean protein, essential amino acids, and essential fatty acids, and contains a large amount of minerals such as iron, phosphorus, and potassium as well as physiologically active substances which are functional components such as isoflavones, saponins, and phytic acid. Recently, studies have been actively conducted to fortify functionality of food and develop functional materials derived from food by elucidating various bioregulatory functions of food ingredients.

In the case of soy milk, studies have been conducted to improve functionality by producing peptides which have nutritional functions that promote digestion and absorption by degrading soybean protein by treating soy milk with proteolytic enzymes as well as physiological activities such as blood pressure enhancement, calcium absorption promotion, anti-allergy, and serum cholesterol reduction. However, studies on the development of functional regulatory biomaterials suitable for processing through enzymatic hydrolysis of soybean proteins and industrialization stages of related materials are still insufficient.

In addition, fermented soy milk is prepared with soybeans as a raw material by undergoing fermentation using microorganisms that are usable in food, and when only the soybeans are consumed as they are, only about 30% is absorbed while the remaining 70% is excreted, whereas when soybeans are fermented, more than 90% of nutrients in soybeans may be absorbed into the body to offer superiority in terms of absorption of nutrients, wherein the fermented soy milk includes not only the nutrients of soybeans but also enzymes such as amylase, protease, lipase, and thrombolytic enzymes produced by microorganisms, in addition to various physiologically active substances such as peptides, amino acids, oligosaccharides, fatty acids, active isoflavones, phytosterols, lecithin, and saponins produced by decomposition of these enzymes. However, problems still remain in the soy milk in regard to soybean odor which is a specific peculiar smell, along with an unpleasant feeling due to rough texture, unsatisfactory consumer preferences in terms of taste that the unique taste of soy milk disappears due to fermentation, and coagulation of a product or strange taste due to heat treatment.

### PRIOR ART DOCUMENT

### Patent Document

Korea Patent Application Publication No. 10-2011-0027247 (published on Mar. 16, 2011)

### DISCLOSURE OF THE INVENTION

### Technical Goals

In order to solve the above problems, an object of the present disclosure is to provide a method of preparing bioconversion powder with enhanced functionality, and is to provide a method of preparing bioconversion powder using a fermentation broth of a Bacillus polyfermenticus strain, by treating whole soy milk with the fermentation broth in which various degrading enzymes and peptide-synthesizing enzymes derived from the Bacillus polyfermenticus strain are mixed so as to identify that low-molecular-weight amino acids, peptides, and various functional components are formed.

### Technical Solutions

The present disclosure provides a method of preparing bioconversion powder, including: (1) crushing soybeans soaked by being immersed in water; (2) heat-treating the crushed soybeans to obtain whole soy milk; and (3) treating the whole soy milk with a Bacillus polyfermenticus KMU01 (Accession number: KCTC 11751BP) strain, a culture thereof, a fermented product thereof, or a mixture thereof.

In addition, the present disclosure provides a health functional food composition including bioconversion powder prepared by the method of preparing bioconversion powder as an active ingredient.

### Advantageous Effects

According to the present disclosure, by treating whole soy milk with a fermentation broth in which various degrading enzymes and peptide-synthesizing enzymes derived from a Bacillus polyfermenticus strain are mixed to identify that low-molecular-weight amino acids, peptides, and various functional components are formed, it is possible to provide a method of preparing bioconversion powder using the fermentation broth of the Bacillus polyfermenticus strain.

### BRIEF DESCRIPTION OF DRAWINGS

FIG. 1 shows a genetic map of a Bacillus polyfermenticus KMU01 strain and genes of an enzyme group.

FIG. 2 shows results of determining degrees of soybean protein degradation and soybean peptide production of whole soy milk and bioconversion powder.

FIG. 3 is a result of evaluating an antioxidant activity of whole soy milk and bioconversion powder.

### BEST MODE FOR CARRYING OUT THE INVENTION

The terms used herein have been selected from currently widely used general terms as much as possible in consideration of functions herein, but these may vary depending on the intentions or precedents of those skilled in the art, the emergence of new technologies, and the like. In addition, in specific cases, there are terms arbitrarily selected by the applicant, and in this case, the meaning will be described in detail in the description of the disclosure. Therefore, the terms used herein should not be defined as simple names of terms, but based on the meaning of the term and the overall contents of the present disclosure.

Unless otherwise defined, all terms used herein, including technical or scientific terms, have the same meaning as commonly understood by those skilled in the art to which the present disclosure pertains. Terms such as those defined in commonly used dictionaries should be construed as having meanings consistent with the meaning in the context of the relevant art and are not to be construed in an ideal or overly formal meaning unless clearly defined in the present application.

The numerical range includes the numerical value defined in the above range. All maximum numerical limits given herein include all lower numerical limits as clearly stated on the lower numerical limits. All minimum numerical limits given herein include all higher numerical limits as clearly stated on the higher numerical limits. All numerical limits given herein will include all better numerical ranges within a wider numerical range as clearly stated on narrower numerical limits.

Hereinafter, the present disclosure will be described in more detail.

As such, as a result of endeavor to develop a method of preparing bioconversion powder with enhanced functional and nutritional properties, the present inventors completed the present disclosure by optimizing conditions for enzymatic hydrolysis of whole soy milk using an enzyme group in which various proteolytic enzymes and peptide-synthesizing enzymes that are secreted and produced by GRAS fermented food-derived microorganisms isolated from fermented foods are mixed and investigating functionality of hydrolysates of whole soy milk so as to develop bioconversion powder with enhanced functional and nutritional properties and a differentiated preparation method that enables application as powder.

The bioconversion refers to a technology for converting an existing material (substrate) using a biological reaction of microorganisms or enzymes produced by microorganisms, specifically, the bioconversion refers to conversion of soybeans or black soybeans (substrates) using an enzyme group which is a culture supernatant in which various enzymes produced by a Bacillus polyfermenticus KMU01 (Accession number: KCTC 11751BP) strain are included, and products obtained by the above bioconversion is called bioconversion powder.

The present disclosure provides a method of preparing bioconversion powder, including: (1) crushing soybeans soaked by being immersed in water; (2) heat-treating the crushed soybeans to obtain whole soy milk; and (3) treating the whole soy milk with a Bacillus polyfermenticus KMU01 (Accession number: KCTC 11751BP) strain, a culture thereof, a fermented product thereof, or a mixture thereof.

The Bacillus polyfermenticus KMU01 (Accession number: KCTC 11751BP) strain was registered in Korean Collection for Type Cultures (KCTC) on Aug. 25, 2010 under the name of Bacillus amyloliquefaciens Kimchi, but was later renamed as Bacillus polyfermenticus KMU01 on Jun. 27, 2018 as the precise species name thereof was identified as Bacillus polyfermenticus.

The soy milk may include all soy milk obtained by conventional methods, and soy milk in the commercial market may be used. For example, a liquid obtained by grinding steamed soybeans swelled by immersing peeled soybeans or skimmed soybeans in water may be used, and a liquid obtained by removing dregs from the liquid may be used, but are not limited thereto. In addition, a solution in which whole soybean flour or skimmed soybean flour is dissolved may be used, soy milk with solid soybean content of 8.0% or more may be used according to the JAS standard, one with solid soybean content of 6.0% or more may be used for soy milk preparation, or one with solid soybean content of 4.0% or more may be used as soy milk drink, but are not limited in terms of solid soybean content.

The Bacillus polyfermenticus KMU01 (Accession number: KCTC 11751BP) strain, culture thereof, fermented product thereof, or mixture thereof may be treated to the whole soy milk at a concentration of 3% (v/v) to 7% (v/v), preferably 5% (v/v).

The Bacillus polyfermenticus KMU01 (Accession number: KCTC 11751BP) strain, culture thereof, fermented product thereof, or mixture thereof may be treated at 35° C. to for 3 to 5 hours, preferably at 37° C. for 4 hours.

The culture may be an artificial medium obtained by culturing the Bacillus polyfermenticus KMU01 (Accession number: KCTC 11751BP) strain, and the fermented product may be a natural medium fermented using the Bacillus polyfermenticus KMU01 (Accession number: KCTC 11751BP) strain.

The artificial medium may be a commercially prepared synthetic medium capable of culturing Bacillus polyfermenticus and bacteria, such as tryptic soy broth (TBS), tryptic soy broth (TSB), nutrient broth (NB), and Luria-Bertani broth (LB), but is not limited thereto.

The natural medium refers to a natural product that may be fermented by bacteria and may be a medium using natural products such as potatoes, tomatoes, and milk, but is not limited thereto.

The culture and the fermented product may exhibit activities of protease, gamma-glutamyltransferase (GGT), and nattokinase.

The protease is an enzyme that hydrolyzes peptide bonds between amino acids constituting a protein with the proteolytic enzyme, some of which include exopeptidases that cleave amino terminus (aminopeptidase) or carboxy terminus (carboxypeptidase) of a protein as well as endopeptidases (e.g., trypsin, chymotrypsin, pepsin, papain, elastase) that cleave the middle of a protein. The gamma-glutamyltransferase (GGT) is an enzyme that transfers the glutamyl group in a gamma-glutamyl compound to a suitable receptor (amine) and is a type of transacylases. The nattokinase, a thrombolytic enzyme produced by Bacillus nano during growth by ingesting nutrients of soybeans when fermenting soybeans, includes vitamin B complex and a large amount of antioxidant enzymes.

The bioconversion powder may exhibit an antioxidant activity.

In addition, the present disclosure provides a health functional food composition including bioconversion powder prepared by the method of preparing bioconversion powder as an active ingredient.

The present disclosure may be generally used as a commonly used food product.

The food composition of the present disclosure may be used as a health functional food. The term “health functional food” as used herein refers to food manufactured and processed with raw materials or ingredients having useful functionality for the human body in accordance with the Health Functional Food Act, and the term “functionality” as used herein refers to the intake to derive effectiveness in health care such as regulation of nutrients or physiological actions for the structure and function of the human body.

The food composition of the present disclosure may include common food additives, and the suitability as the “food additive” is determined by the standards and criteria related to corresponding items according to the general rules and general test methods of Korean Food Additives Codex approved by the Ministry of Food and Drug Safety, unless otherwise stipulated.

The items listed in the “Korean Food Additives Codex” may include, for example, chemically synthesized compounds such as ketones, glycine, potassium citrate, nicotinic acid, and cinnamic acid, natural additives such as persimmon color, licorice extracts, crystallized cellulose, kaoliang color, and guar gum, and mixed preparations such as sodium L-glutamate preparations, noodle-added alkali agents, preservative agents, and tar color agents.

The food composition of the present disclosure may be manufactured and processed in the form of tablets, capsules, powder, granules, liquids, and pills.

For example, hard capsule preparations among health functional foods in the form of capsules may be prepared by mixing and filling the composition according to the present disclosure in conventional hard capsules along with additives such as excipients, and the soft capsule preparations may be manufactured by mixing the composition according to the present disclosure with the additives such as excipients and then filling the same in capsule bases such as gelatin. The soft capsule preparations may include, if necessary, plasticizers such as glycerin or sorbitol, colorants, and preservatives.

The definition of terms for the excipient, binder, disintegrant, lubricant, flavor enhancer, and flavoring agent is described in documents known in the art and includes those having the same or similar functions. The type of food is not particularly limited and includes all health functional foods in the ordinary sense.

### MODES FOR CARRYING OUT THE INVENTION

Hereinafter, example embodiments will be described in detail to help the understanding of the present disclosure. However, the following example embodiments are merely illustrative of the content of the present disclosure, and the scope of the present disclosure is not limited to the following examples. The example embodiments of the present disclosure are provided to more completely explain the present disclosure to those of ordinary skill in the art.

### Example 1. Evaluation on Various Activities of Enzymes Derived from Fermented Food Microorganisms

To prepare functional fermented whole soy milk, various enzymatic activities of a Bacillus polyfermenticus KMU01 (Accession number: KCTC 11751BP) strain, a fermentation strain, were evaluated.

First, the activities of protease, gamma-glutamyltransferase (GGT), and nattokinase (Nattokinase) were evaluated. The fermentation strain was inoculated in 50 mL of tryptic soy broth (TSB) medium and cultured at 37° C. for 24 hours, and a supernatant of the culture was collected and centrifuged at 8,000 rpm for 20 minutes. Supernatants of centrifuged culture were used to evaluate each enzymatic activity.

For the activity of the protease, 0.1 ml of 0.5% azocasein solution and 0.1 ml of coenzyme solution were added to an eppendorf tube as substrates, a reaction was performed in a constant temperature water bath at 37° C. for 1 hour, and then 0.4 ml of 10% trichloroacetic acid solution was added to stop the reaction. The reaction solution was centrifuged at 13,000 rpm for 5 minutes to collect the supernatant which was then neutralized by adding 0.6 ml of N NaOH solution to 0.6 ml of the supernatant, the absorbance was measured at 420 nm, and the activity of protease was evaluated by setting, as 1 unit, an amount of enzymes that free 1 μg of tyrosine for 1 minute under the reaction condition.

For the activity of gamma-glutamyltransferase (GGT), 0.01 ml coenzyme solution and 0.09 ml of 50 mM phosphate buffer solution (pH 7.0) containing 0.1 mM γ-L-glutamyl-p-nitroaniline (p-NA-Glu, Sigma-Aldrich) were mixed, a reaction was performed at 40° C. for 30 minutes, and then 0.01 ml of 3.5 N acetic acid was added to stop the reaction. The amount of free p-nitroaniline was measured at 410 nm. Using p-nitroaniline as a standard solution, the enzymatic activity was calculated by drawing a standard curve. For 1 unit of enzymatic activity of GGT, a degree of enzymatic activity of GGT was evaluated by calculating the amount of enzyme that frees 1 mole of p-nitroaniline from p-NA-Glu per minute.

For the activity of nattokinase, 350 μl of 50 mM borate buffer (pH 8.5), 100 μl of 1% fibrinogen solution, and 25 μl of 10 unit thrombin solution were mixed, a reaction was performed at 37° C. for 10 minutes, and then 25 μl of coenzyme solution was added, followed by a reaction at 37° C. for 1 hour. 500 μl of 0.2 M TCA solution was added to the reaction solution to stop the reaction, and then the mixture was allowed to stand at 37° C. for 10 minutes. After collecting the supernatant by centrifuging the reaction solution at 8,000 rpm for 20 minutes, the absorbance of the collected supernatant was measured at 275 nm, and the enzymatic activity was calculated according to the following calculation formula to evaluate a degree of the thrombolytic activity.

Degree of thrombolytic activity (FU/ml)=A1−A0/0.01×1/60×1/0.025×D
A1: Absorbance value of a sample
A0: Absorbance value (blank) of a blank test sample prepared without addition of coenzyme solution
0.01: Activity of enzymes with absorbance increased by 0.01 per minute
60: Enzyme reaction time (min)
0.025: Amount of enzymes used
D: Dilution rate of sample

As shown in Table 1 below, protease activity was found to be 78 U/ml, GGT activity was 3500 mU/ml, and nattokinase activity indicating the hemolytic activity was 24 U/ml.

TABLE 1

| Evaluation on enzymatic activity | B. polyfermenticus KMU01 |
|---|---|
| Protease activity (U/ml) | 78 |
| GGT activity (mU/ml) | 3500 |
| Nattokinase activity (U/ml) | 24 |

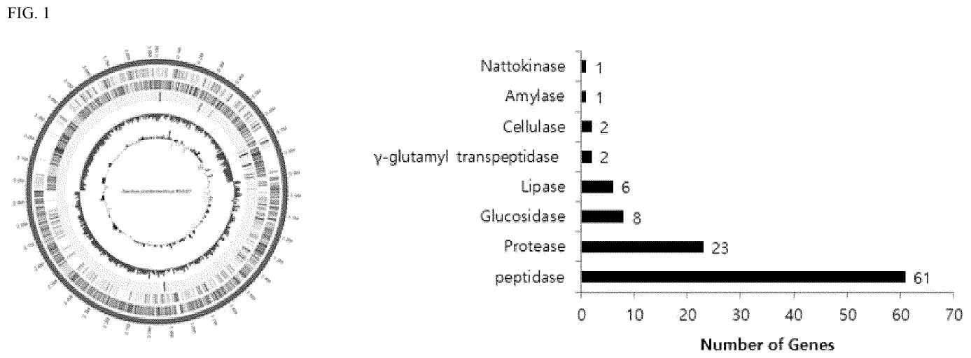

In addition, the genome of the Bacillus polyfermenticus KMU01 (Accession number: KCTC 11751BP) strain was analyzed with PacBio_20K sequencer and SMRT 2.3.0 (HGAP2) assembler to identify genes of various functional enzymes. As a result, as shown in FIG. 1, it was determined that the Bacillus polyfermenticus KMU01 (Accession number: KCTC 11751BP) strain has 61 peptidase genes, 23 protease genes, 8 glucosidase genes, 6 lipase genes, 2 γ-glutamyl transpeptidase (GGT) genes, 2 cellulase genes, amylase genes, and nattokinase genes.

### Example 2. Preparation of Functional Bioconversion Powder Using the Fermentation Strain

Used as an enzyme solution for bioconverting whole soy milk was a supernatant obtained by culturing the Bacillus polyfermenticus KMU01 (Accession number: KCTC 11751BP) strain in tryptic soy broth (TSB) medium at 37° C. for 24 hours. In order to prepare the whole soy milk, cv. Socheongja from Iksan was washed and immersed in water for 14 hours, and then water was removed, followed by grinding using a grinder while removing water. The ground sample was boiled at 100° C. for 30 minutes, and then whole soy milk was obtained. The obtained whole soy milk was treated with the enzyme solution at a rate of 5% (v/v), a reaction was performed at 37° C. for 4 hours for bioconversion, and lyophilization was carried out to prepare the bioconversion powder.

TABLE 2

| Classification | Conditions for bioconversion |
|---|---|
| Whole soy milk (mL) | 95 |
| Enzyme solution (mL) | 5 |
| Reaction temperature (° C.) | 37 |
| Reaction time (hr) | 4 |

### Example 3. Evaluation on a Degree of Hydrolysis of the Bioconversion Powder

A degree of hydrolysis of the bioconversion powder prepared in Example 2 for proteins was evaluated. 2 mL of hydrolysates from each sample were taken, added to a test tube containing 2 mL of 20% (w/v) trichloroacetic acid (TCA), and then centrifuged (3,000×g, after mixing, and a certain amount of centrifuged supernatant was taken to measure an amount of protein and calculate the degree of hydrolysis. As a result of calculation, it was found that the degree of hydrolysis of the bioconversion powder was 53.8%.

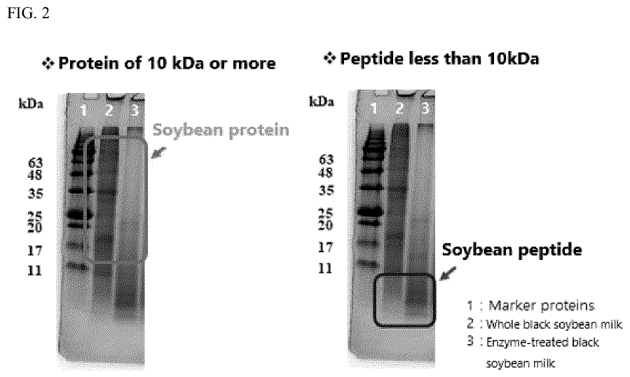

In addition, as a result of identifying a difference in molecular weight of the black soybean protein by performing 10% sodium dodecyl sulfate polyacrylamide gel electrophoresis (SDS-PAGE), as shown in FIG. 2, compared to a control group (whole black soybean milk), the content of soybean peptides of 10,000 Da or less was increased 1.23 times in the bioconversion powder (enzyme-treated black soybean milk).

### Example 4. Analysis on a Composition of Amino Acids in the Bioconversion Powder

An automatic amino acid analyzer (Biochrom 30+) was used to analyze functional amino acids in the bioconversion powder. As shown in Table 3 below, the content of functional amino acids such as branched chain amino acids (BCAAs) required for muscle growth and aromatic amino acids which are precursor amino acids of neurotransmitters has been increased.

TABLE 3

Content (mg/L, mg/soybean 70 g)

| | Amino | Whole soy milk | Bioconversion |
|---|---|---|---|
| Classification | acid | (Control group) | powder |
| Aromatic | Phe | 8 | 65 |
| | Tyr | 5 | 28 |
| | Trp | 14 | — |
| BCAA | Val | 8 | 72 |
| | Leu | 3 | 65 |
| | Ile | 1 | 21 |
| Others | GABA | 2 | 12 |
| | Glu | 17 | 9 |
| | Cys | 5 | 14 |
| | His | 8 | 75 |
| | Pro | N/D | 51 |
| | Lys | 3 | 79 |
| | Arg | 110 | 111 |

### Example 5. Analysis on a Composition of the Bioconversion Powder

In order to identify functional components of the bioconversion powder, analysis was conducted on soybean dietary fiber, soybean oligosaccharides, isoflavones, flavonoids, and anthocyanins by requesting to the Korea Functional Food Institute, Korea Analysis Test Researcher, and Korea Basic Science Institute. As shown in Table 4 below, soybean dietary fiber, soybean oligosaccharides (raffinose, stachyose), aglycon isoflavones, flavonoids, and anthocyanins were identified as components of the bioconversion powder.

TABLE 4

| | Soybean | | | | | | |
|---|---|---|---|---|---|---|---|
| | oligosaccharides | | Dietary fiber | | Isoflavones | Flavonoids | Anthocyanins |
| Product name | (mg/g) | | (mg/g) | | (mg/g) | (mg/g) | (mg/g) |
| Bioconversion | Raffinose | 4.3 | Method 1 | 131 | 0.67 | 20.38 | 3.49 |
| powder | | | (including | | | | |
| | | | insoluble) | | | | |
| | Stachyose | 14.8 | Method 2 | 267 | | | |
| | | | Low- | | | | |
| | | | molecular- | | | | |
| | | | weight water- | | | | |
| | | | soluble | | | | |
| | | | (Trisaccharide | | | | |
| | | | or more) | | | | |

In addition, as a result of analyzing G-peptide by the Korea Basic Science Institute, contents of γ-glutamyl glycine (γ-Glu-Gly), γ-glutamyl-valine (γ-Glu-Val), γ-glutamyl-cysteine (γ-Glu-Cys), γ-glutamyl-leucine (γ-Glu-Leu), and γ-glutamyl-glutamine (γ-Glu-Gln), in which functions of increasing taste (kokumi) and alleviating inflammatory bowel disease and inflammation have been reported, increased in the enzyme-treated soy milk as shown in Table 5 below.

TABLE 5

| | | Whole soy | Enzyme-treated | |
|---|---|---|---|---|
| | | milk | whole soy milk | |
| γ-Glutamylpeptides | | (ng/mL) | (ng/mL) | Functions |
| EG | γ-GLU-GLY | 4.690 | 92.989 | Taste (kokumi) |
| EV | γ-GLU-VAL | 2.033 | 7.105 | Taste (kokumi) Alleviation of |
| | | | | inflammatory bowel disease and |
| | | | | intestinal inflammation |
| EC | γ-GLU-CYS | 2.681 | 13.799 | Taste (kokumi) Alleviation of |
| | | | | inflammatory bowel disease and |
| | | | | intestinal inflammation |
| EL | γ-GLU-GLN | 0.568 | 7.241 | Taste (kokumi) |
| EQ | γ-GLU-GLU | 0.516 | 57.376 | Taste (kokumi) |

### Example 6. Evaluation on an Antioxidant Activity of the Bioconversion Powder

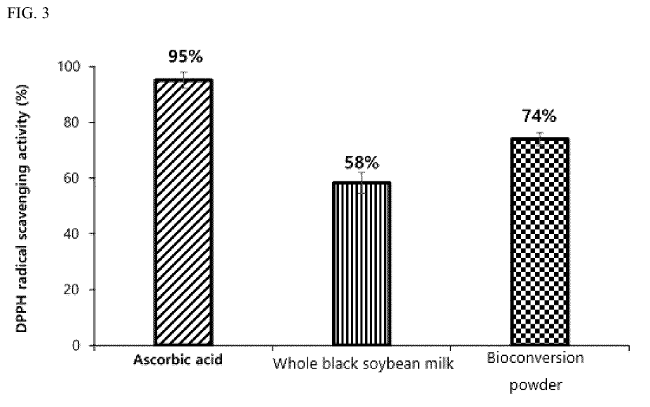

DPPH radical scavenging activity was analyzed to evaluate an antioxidant activity of the bioconversion powder. For DPPH radical scavenging ability, as a method of measuring, using a spectrophotometer, a degree of reduction in DPPH radical by reacting 1.1-diphenyl-2-picrylhydrazyl (DPPH), a stable free radical, with a predetermined sample solution, 50 μl of the sample and 50 μl of 0.1 mM DPPH solution were mixed, the mixture was allowed to stand in a dark room at room temperature for 30 minutes, and then the absorbance was measured at 517 nm to calculate the degree of radical reduction compared to the control group. Blank absorbance was measured by mixing 50 μl of water and 50 μl of 0.1 mM DPPH solution, and the control absorbance of each sample was measured by mixing 50 μl of the sample and 95% ethanol. As a sample of a positive control group, ascorbic acid was used. As shown in FIG. 3, high DPPH radical scavenging activity of 74% was shown in the bioconversion powder compared to the control group (whole black soybean milk).

As described above, a specific part of the content of the present disclosure is described in detail, for those of ordinary skill in the art, it is clear that the specific description is only a preferred embodiment, and the scope of the present disclosure is not limited thereby. In other words, the substantial scope of the present disclosure may be defined by the appended claims and their equivalents.

1. A method of preparing bioconversion powder, the method comprising:
(1) crushing soybeans soaked by being immersed in water;
(2) heat-treating the crushed soybeans to obtain whole soy milk; and
(3) treating the whole soy milk with a Bacillus polyfermenticus KMU01 (Accession number: KCTC 11751BP) strain, a culture thereof, a fermented product thereof, or a mixture thereof.

2. The method of claim 1, wherein the Bacillus polyfermenticus KMU01 (Accession number: KCTC 11751BP) strain, culture thereof, fermented product thereof, or mixture thereof is treated at a concentration of 3% (v/v) to 7% (v/v).

3. The method of claim 1, wherein the Bacillus polyfermenticus KMU01 (Accession number: KCTC 11751BP) strain, culture thereof, fermented product thereof, or mixture thereof is treated at 35° C. to 40° C. for 3 to 8 hours.

4. The method of claim 1, wherein the culture is an artificial medium obtained by culturing the Bacillus polyfermenticus KMU01 (Accession number: KCTC 11751BP) strain.

5. The method of claim 1, wherein the fermented product is a natural medium fermented using the Bacillus polyfermenticus KMU01 (Accession number: KCTC 11751BP) strain.

6. The method of claim 1, wherein the culture and the fermented product exhibit activities of protease, gamma-glutamyltransferase (GGT), and nattokinase.

7. The method of claim 1, wherein the bioconversion powder exhibits an antioxidant activity.

8. A health functional food composition, comprising bioconversion powder prepared by the method of claim 1 as an active ingredient.
